(19) United States
(12) Patent Application Publication
(10) Pub. No.: US 2005/0004488 A1
(43) Pub. Date: Jan. 6, 2005

(54) Portable VO2 meter

(75) Inventors: John Hoppe, Ventura, CA (US); Andrew Huszcza, Ventura, CA (US)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Vacumetrics Inc.

(21) Appl. No.: 10/851,237

(22) Filed: May 24, 2004

(30) Foreign Application Priority Data
May 27, 2003 (CA) 2,430,613

(51) Int. Cl. A61B 5/08

(52) U.S. Cl. 600/529

(57) ABSTRACT

A method of determining VO2 for an individual from known or predicted RQ values, for example at RQ≅1; (i) providing an oxygen sensor, (ii) providing preferably bi-directional, respiratory flow sensing, (iii) determining the onset of the sustained rise of the ventilatory equivalent for oxygen, Ve/VO2, (iv) providing calculated VO2 values with RQ-based adjustments even in the absence of available CO2 data.

### FIELD OF THE INVENTION

This invention relates to a portable device and related methods of use of said device which provides more accurate VO2 measurement during exercise using an oxygen sensor but no CO2 sensor, which is typically required by known systems and devices.

### BACKGROUND OF THE INVENTION

Abbreviations used within this specification are defined as follows:

| | |
|---|---|
| AT | Anaerobic Threshold (same as LT) |
| CPX | Cardiopulmonary Exercise System |
| HR | Heart rate |
| ΔHR | Change in HR during a fixed time period |
| LT | Lactate Threshold (same as AT) |
| VE | Minute Ventilation (breathing volume per minute) |
| R, RQ, RER | Respiratory Quotient or Respiratory Exchange Ratio |
| TBF | TurboFit software |
| TT | Vista TurboTrainer ™ |
| VCO2 | CO2 output |
| VO2 | Oxygen consumption |
| ΔVO2 | Change in VO2 during a fixed time period |

Those skilled in the art will appreciate the meaning of the above terms and each complete definition is therefore not provided.

Metabolic Measurement Systems, also known as Metabolic Carts, Cardiopulmonary Exercise Systems (CPX), VO2 Measurement Systems, and in Europe, Ergospirometry Systems, are used to measure the oxygen consumption (VO2), CO2 output (VCO2) and breathing volume (VE) in clinical health assessment, fitness training and exercise prescription.

During a CPX test Oxygen Uptake Response is measured. Determination of VO2max is the gold standard to measure functional capacity of the cardiovascular system to transport oxygen. Values of VO2max depend on the mode of exercise, the degree of training and the integrity of cardiovascular function. It is usually reduced in any sort of cardiopulmonary disease. In most cases, except in athletes, the presence of a normal or elevated VO2max virtually ensures the absence of any major cardiovascular or pulmonary diseases.

VO2max is expressed in ml/kg of body weight and relates to exercise tolerance. Knowing the unique anaerobic threshold can be used to design a workout plan that will improve fitness and maximize calories burned. Measurement of VO2max or PeakVO2 will provide a true assessment of fitness level.

CPX systems normally combine an oxygen sensor, a CO2 sensor and a method of measuring breathing volume. The resulting data is fed to a computer containing appropriate software. The assignee has produced a series of such CPX systems under the trade name “Vista™” and has written several software versions for PC's under the trade name “TurboFit™”. Please refer to the a website www.Vacumed.com for more details in this regard, said contents of the website as of the priority date of filing this application being hereby incorporated by reference.

Others in the field have also endeavored to produce CPX systems without a CO2 sensor, but such systems lack the accuracy of data that a CO2 analyzer can provide. For example an Italian company, Cosmed, and an American company, Korr offer such devices without CO₂ sensors. For example the Cosmed model “K2” is a portable, battery-operated device with radio signal transmission of “live” data. But it assumes that the RQ must equal 1, which means the resulting VO₂ will only be correct when RQ is 1. The resulting errors in VO2 result in approximation only of the VO2 level when RQ does not equal 1.

A number of variables can be calculated from analyzing exhaled breaths, among them “RQ” from the formula
RQ=VCO2/VO2. (Equation 1)

This relationship demonstrates the importance of knowing VCO2 in order to measure VO2 by traditional methods and devices. But a CO2 analyzer required to determine VCO2 is costly and has a typical high power consumption. An O2 analyzer is typically less expensive.

All typical CPX's (VO2 Measurement Systems) contain similar basic building blocks; namely oxygen and CO2 analyzers, a ventilation measurement device, interfacing electronics, gas sampling system, data acquisition system and some kind of computer and software.

The final and perhaps most important component is the software. It is the software that ultimately determines the accuracy of the VO2 measurement. Even when top end gas analyzers are utilized, if the software does not correctly align gas data with flow data, or if the compensation for barometric pressure, temperature and humidity is not handled correctly, then the system cannot report accurate VO2's.

None of the prior art constructions identified above known to Applicants addresses the issues which the current invention focuses in upon, namely improving the accuracy of VO2 meters which operate without a CO2 analyzer. With all of the knowledge of those designing VO2 meters none of the inventors including Applicant's prior construction take advantage of the ease in determining VO2 from measurements including O2 which allows for simplicity of determination of V02. Nowhere within the prior art is such a device known to applicants' knowledge.

It is therefore a primary object of the invention to provide a VO2 meter to determine VO2 without a CO2 analyzer and yet providing accurate output.

It is yet another object of this invention to provide such a device which is portable.

It is another object of the invention to make such a device affordable.

It is yet a further object of the invention to provide a VO2 meter which accumulates data over an extensive operating period which when desired may be uploaded to a PC.

It is yet a further object of the invention to provide a method of measuring VO2 accurately without measuring CO2 content of expired gases.

It is yet a further object of the invention to provide a method of measuring VO2 which is cost effective.

Further and other objects of the invention will become apparent to those skilled in the art when considering the following summary of the invention and the more detailed description of the preferred embodiments illustrated herein.

### SUMMARY OF THE INVENTION

The present invention therefore provides
(i) a method of computing VCO2 from known or predicted RQ;
(ii) a method of correcting VO2 with RQ-based corrections in the absence of CO2 data;
(iii) a device with RQ-compensating software that provides more accurate VO2 measurement using only an oxygen sensor (no CO2 sensor) by re-computing (compensating) VO2 values with either known or predicted “RQ”; and
(iv) a method of stabilizing the O2 sensor calibration to compensate for changes in barometric pressure and/or altitude using automatic barometric pressure compensation implemented by means of continuous measurement of barometric pressure with electronic method and using the resulting signal in the gain feedback loop of corresponding O2 amplifiers to maintain constancy of the O2 readings at any given calibration value.

To these ends according to a primary aspect of the invention there is provided a method of determining VO2 from known or predicted RQ values, for example at RQ≅1 using, preferably bi-directional, respiratory flow sensing and the onset of the sustained rise of the ventilatory equivalent for oxygen, Ve/VO2, thereby providing VO2 values with RQ-based adjustments in the absence of available CO2 data.

According to yet another aspect of the invention there is provided a device with software implementing the above methods that provides more accurate VO2 measurement during exercise using only an oxygen sensor (no CO2 sensor).

According to yet another aspect of the invention there is provided a VO2 meter comprising a patient interface, an O2 sensor, a flow (volume) meter, (in one embodiment bi-directional) a heart rate pickup device, for example by “Polar”, a mixing chamber, (preferably a mini-mixing chamber), temperature and barometric pressure sensors, a memory and a PC interface such as a USB. Preferably the USB memory and interface may further comprise up to 90 minutes of breath-by-breath data, to be downloaded to a PC for further evaluation. Preferably the meter is portable.

According to yet another aspect of the invention there is provided a method of determining VO2 for an individual from known or predicted RQ values, for example at RQ≅1;
(i) providing an oxygen sensor,
(ii) providing preferably bi-directional, respiratory flow sensing,
(iii) determining the onset of the sustained rise of the ventilatory equivalent for oxygen, Ve/VO2,
(iv) providing calculated VO2 values with RQ-based adjustments even in the absence of available CO2 data.

According to one embodiment of the invention there is provided a device for determining VO2 from known or predicted RQ values for implementing the above mentioned method comprising,
an oxygen sensor but lacking a CO2 sensor,
a respiratory flow sensor,
and computer related interfaces and software
   thereby determining accurate VO2 measurement for an individual during exercise.

According to yet another aspect of the invention there is provided a device for determining VO2 from known or predicted RQ values comprising,
an oxygen sensor but lacking a CO2 sensor,
a respiratory flow sensor,
   and computer related interfaces and software
thereby determining accurate VO2 measurement for an individual during exercise. Preferably said meter is portable.

According to yet another aspect of the invention there is provided a VO2 meter for determining VO2 from known or predicted RQ values comprising,
a patient interface,
an O2 sensor,
a flow (volume) meter, (in one embodiment bi-directional)
a heart rate pickup device, for example by “Polar”,
a mixing chamber, (preferably a mini-mixing chamber),
temperature and
barometric pressure sensors,
a memory and a PC interface such as a USB
   thereby determining accurate VO2 measurement for an individual during exercise. Preferably said memory and interface further comprise up to 90 minutes of breath-by-breath data, to be downloaded to a PC for further evaluation. In one embodiment said meter is portable.

### BRIEF DESCRIPTION OF THE DRAWINGS

FIG. 1 is a schematic diagram generally showing the components of the portable VO2 meter illustrated in a preferred embodiment of the invention.

### DETAILED DESCRIPTION OF THE INVENTION

The TurboFit Software for VO2 Measurement Systems will be running on a PC to receive and analyze the data from the portable VO2 meter.

The Software Features include the following:
Optional input of personal test subject information, such as smoking, alcohol and exercise habits, resting spirometry, blood pressure and lipids values. Heart rate import options from EKG's, Polar watch, and or selected exercise devices.
Auto-calibration feature for fast, error resistant calibration. Predicted values shown on-screen during test and on print-out. Fitness Analysis Report includes bar graphs of VO2 actual vs. predicted, heart rate and breathing reserve; personalized lactate threshold-based training heart rate range and recommended weight loss.
Printed explanation of test and results to hand to test subject.
Trend Analysis: Example, test a subject several times during a training period, then plot his Lactate Threshold, VO2(peak) or anything else over time. Teach how ambient conditions affect O2.
Special interactive display allows variance of temperature, humidity and barometric pressure to determine the resulting exact O2 concentration.
Expanded Graphics Capability is provided for example the ability, to plot up to 8 variables in a window, to select color, line thickness, symbol shape and fill for each variable. In this way the important variables are emphasized.
Troubleshooting features include an oscilloscope signal display allowing variance and filtering of a resulting signal, and includes extensive fault detection algorithms.
Custom reports may be provided based on pre-configuration of up to 6 report groups, such as reports for VO2 peak testing.

The device is, in one embodiment portable, but not in all embodiments of the invention ,and each embodiment requires heart rate monitoring. This may be achieved a number of ways but to enhance portability the Polar monitor is used. The “Polar” Heart Rate Watch includes a wristband display with battery, lightweight electrode chest belt and transmitter. The reader is referred to the product literature in this regard or the website, the contents of which is hereby incorporated by reference. For example the Polar Vantage NV™ is ideal for athletic training, coaching, research and physical education. Also for the competitive athlete. With an available interface, and all stored heart rate data can be downloaded to a PC for the complete package for analyzing, tracking and programming any training program.

Some of the features of the invention include but are not limited to the explanations that follow. Three programmable target zones are provided with out-of-zone alarms displaying heart rate, lap time and elapsed time or time of day and calculating the time spent above, below and within target heart rate zone. The system calculates the average and maximum heart rate for total file lap or split time indicating the average heart rate of the preceding lap. The system also calculates recovery heart rate or recovery time and automatic recording of heart rate at 5, 15, or 60 second intervals and may include three programmable interval timers including countdown for 133 hours with unlimited files for recording information. The invention also includes the R-R intervals for heart rate information every 5, 15, 60 seconds, and displays relaxation rate. The information may be coded during transmission to avoid crosstalk caused by other users.

This disclosure describes the development by the inventors of a portable device that measures VO2 without a CO2 sensor, especially during exercise. It can be operated on-line while connected to a PC or store data in memory to be downloaded after a test. The reason for eliminating the CO2 sensor is its cost and usually high power consumption.

The present device will contain a patient interface, an O2 sensor, bi-directional flow (volume) meter, a heart rate pickup device, for example by “Polar”, a mini-mixing chamber, temperature and barometric pressure sensors, memory and USB download. An existing USB I/F is to be modified to store up to 90 minutes of breath-by-breath data, then use TurboFit's IMPORT option to download the data to a PC.

It should be obvious that VCO2 can be calculated if RQ is known, thereby making a system without CO2 sensor much more accurate. Therefore, if equation 1 is solved for VCO2, then
VCO2=RQ×VO2 (Equation 2)

It is known that normal resting RQ is about 0.85, and RQ at the AT or LT=1.

RQ increases further if workload, and therefore heart rate, increases. Published data recommends that in order to prove that an exercising person has reached his upper limit of VO2 (also known as VO2max), that persons age-predicted maximum heart rate should be achieved and his/hers RQ should reach 1.10 or higher.

Heart rate at rest is assumed to be 70, unless measured more accurately. The maximum heart rate that can be attained during exercise is calculated
220−age=HRmax (Equation 3)

The LT (AT) of people of normal fitness level occurs at 60% of VO2max (or HRmax), therefore the LT of people of normal fitness level can be calculated according to the following formula:
LT=(220−age−HRrest)×% HRmax+HRrest (Equation 4)

The software will then prepare a table of HR vs. RQ, for a 50-year old which will be similar to the following:

| HR | RQ |
|---|---|
| 70 | .80 |
| 100 | .90 |

The software will interpolate all HR between rest and max.

| | |
|---|---|
| 130 | 1 |
| 160 | 1.10 |

Software will calculate a straight line increase of RQ with increasing HR.

Manual override of HRrest and HR at LT, if known or determined by other means may well provide more accurate corrections.

Further Refinements:

It is known that athletes and super athletes has lower than average resting heart rates and their LT (AT) occurs at higher than 60% of VO2max. Therefore, a software input of known or perceived fitness status can further refine the predicted RQ and thus the VO2 correction.

Estimated or known fitness level to predict the LT.

| Fitness Level | HRrest | Predicted LT |
|---|---|---|
| Average Fitness: | 70 | LT = 60% of HRmax. |
| Above Average Fitness: | 60 | LT = 70% of HRmax |
| Athletic Fitness level: | 50 | LT = 80% of HRmax |

Proposed Software Operation for Data Transfer

1. Connect USB cable between VO2 meter and PC.

2. In Turbofit REVIEW MENU, click IMPORT. Add new option:
“Import TURBOTRAINER DATA”

3. Display files stored in TurboTrainer or automatically transfer them to the software TBF.

4. Select a file to be processed, open TURBOTRAINER PROCESSOR MENU.
This Menu will contain 3 frames: A patient data frame, a processing data frame and a graphic window showing VO2, RQ computed from the default HR settings, HR and watts (zeroes if no watt data).

5. The Processor Data frame will show default fitness level (average), default resting HR of 70 (until another fitness level is selected or HRrest is entered manually), default HR at LT (set to 120 until patient DOB, age, is entered, then computed according to equations 5), default RQ at rest of 0.85 (may be manually modified but limited to 0.70 to 0.99) and a small sub-frame offering the choice of (*) Default Heart Rate or (*) Manual Entry Heart Rate.

6. After patient data is entered TBF will create a default file name, which may be modified by the user.

7. The graphic display shall have two cursors:
One, to set the start (prior to which may be cal gas). User may change position of Start cursor. Data prior to Start cursor will not be saved after FINISH.
Two, to show the LT, this 2nd cursor is moved to that position where the HR calculation of the LT first equals RQ of 1. So if equation 5 has calculated the LT to be at a HR of 120, then show the 2nd cursor where HR equals 120 the first time it reaches that level (it may reach or cross that HR several times, but the cursor only shows where it first crosses). Changing the HR in the Processor Data frame recalculates the LT and moves the cursor.

8. VO2 graph is updated every time an entry is modified, such as RQrest, HRrest or HR at LT.

9. When all data is entered, user has two options:
a. Click on FINISH button. This closes Processor menu, saves the changes in the newly named TBF file, opens the existing REVIEW menu with its existing Control Panel for further processing or printing.
b. Click on PROCESS NEXT. This saves the just processed file under the newly named TBF file for later access through the normal REVIEW menu and opens the previous TURBOTRAINER IMPORT window to process the next file.

10. The Processor Menu will have a CLEAR MEMORY button that erases the TurboTrainer memory after all data files have been processed.

11. Occasionally it may be necessary to re-process a TurboTrainer file, such as when resting HR or LT-HR may need to be modified.
To do so, the EDIT DATA button password “REPROCESS” should allow re-entry into the TURBOTRAINER PROCESSOR MENU and allow changes.

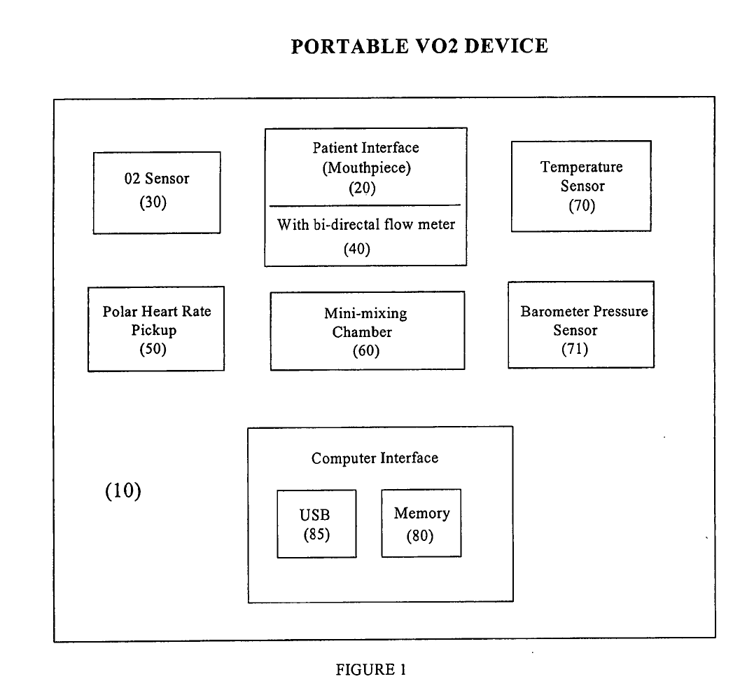

Referring to FIG. 1 there is illustrated in schematic form the portable VO2 meter of the present invention 10 having a patient interface 20, an O2 sensor 30, a bi-directional flow (volume) meter 40, a heart rate pickup device 50, a mini-mixing chamber 60, temperature and barometric pressure sensors 70, 71, a memory 80 and a PC interface such as a USB 85. The memory 80 and interface 85 includes up to 90 minutes of breath-by-breath data, to be downloaded to a PC for further evaluation.

The present device and its TurboFit software will therefore correct the measured VO2 (calculated without CO2 sensor data) and apply the following corrections:
1. The RQ at the start of exercise (rest) is assumed to be 0.85.
2. The RQ at the AT/LT is known to be 1.00.
3. The RQ at the age-predicted maximum heart rate is assumed to be 1.10.
4. The software will interpolate a straight-line RQ correction that will vary between 0.85 at rest and 1.00 at the AT/LT and calculate an estimated VCO2 according to equation 2 above.
5. The software will interpolate a separate (the slope is likely to be different) straight-line RQ correction that will vary between 1.00 at the AT/LT and 1.10 at the age-predicted maximum heart rate and again calculate an estimated VCO2 according to equation 2 above. The corrected VO2 will then be calculated using standard software formulas.

As many changes can be made to the preferred embodiments of the invention without departing from the scope thereof. It is intended that all matter contained herein be considered illustrative of the invention and not it a limiting sense.

1. A method of determining VO2 for an individual from known or predicted RQ values, for example at RQ≅1;
(i) providing an oxygen sensor,
(ii) providing preferably bi-directional, respiratory flow sensing,
(iii) determining the onset of the sustained rise of the ventilatory equivalent for oxygen, Ve/VO2,
(iv) providing calculated VO2 values with RQ-based adjustments even in the absence of available CO2 data.

2. A device for determining VO2 from known or predicted RQ values for implementing the method of claim 1 comprising,
an oxygen sensor but lacking a CO2 sensor,
a respiratory flow sensor,
and computer related interfaces and software
thereby determining accurate VO2 measurement for an individual during exercise.

3. A device for determining VO2 from known or predicted RQ values comprising,
an oxygen sensor but lacking a CO2 sensor,
a respiratory flow sensor,
and computer related interfaces and software
thereby determining accurate VO2 measurement for an individual during exercise.

4. A VO2 meter for determining VO2 from known or predicted RQ values comprising, a patient interface,
an O2 sensor,
a flow (volume) meter, (in one embodiment bi-directional)
a heart rate pickup device, for example by “Polar”,
a mixing chamber, (preferably a mini-mixing chamber),
temperature and
barometric pressure sensors,
a memory and a PC interface such as a USB
thereby determining accurate VO2 measurement for an individual during exercise.

5. The meter of claim 4 wherein said memory and interface further comprise up to 90 minutes of breath-by-breath data, to be downloaded to a PC for further evaluation.

6. The meter of claim 3 wherein said meter is portable.

7. The meter of claim 4 wherein said meter is portable.

8. A VO2 meter comprising a patient interface, an O2 sensor, a bi-directional flow (volume) meter, a heart rate pickup device, a mini-mixing chamber, temperature and barometric pressure sensors, a memory and a PC interface such as a USB thereby determining accurate VO2 measurement for an individual during exercise.

9. The meter of claim 8 wherein the USB memory and interface further comprise up to 90 minutes of breath-by-breath data, to be downloaded to a PC for further evaluation.

10. The meter of claim 8 wherein said meter is portable.
